(19) United States
(12) Patent Application Publication
(10) Pub. No.: US 2013/0203181 A1
(43) Pub. Date: Aug. 8, 2013

(54) DETERMINATION OF INTERACTIONS BETWEEN A SUBSTANCE OR A SUBSTANCE MIXTURE AND A TARGET

(71) Applicant: Hinnerk Boriss, Leipzig (DE)

(72) Inventor: Hinnerk Boriss, Leipzig (DE)

(73) Assignee: SOVICELL GMBH, Leipzig (DE)

(21) Appl. No.: 13/643,838

(22) PCT Filed: Apr. 20, 2011
(86) PCT No.: PCT/EP11/56324

§ 371 Date: Dec. 27, 2012

(30) Foreign Application Priority Data
Apr. 27, 2010 (DE) 10 2010 018 965.0

(51) Int. Cl. G01N 33/543

(52) U.S. Cl. CPC G01N 33/543
USPC 436/501; 422/553

(57) ABSTRACT

A method includes incubating at least a first and a second sample of the substance or of the substance mixture with the target immobilized on a solid carrier. Incubation is in each case effected in a sample container. The first and the second sample are incubated with different amounts of the target, whereas all sample containers include the same amount of buffer solution during incubation. After incubation, the solid carrier is separated from the buffer solutions together with the target immobilized thereon as well as, if applicable, substance bound thereon. The concentration of the substance or of the substance mixture is then measured in the respective supernatant.

### RELATED APPLICATIONS

This is a §371 of International Application No. PCT/EP2011/056324, with an international filing date of Apr. 20, 2011 (WO 2011/134860 A1, published Nov. 3, 2011), which is based on German Patent Application No. 10 2010 018965.0, filed Apr. 27, 2010, the subject matter of which is incorporated herein by reference.

### TECHNICAL FIELD

This disclosure relates to a method for determining interactions between a substance or a substance mixture and a potential target as well as an device which can be used to carry out the method.

### BACKGROUND

Determination of interactions between a substance or a substance mixture and a target is very important in pharmaceutical and pharmacological research. The fields of research both deal with interactions between substances or substance mixtures and living beings. In this case, it is essential to quantify the binding properties of individual substances with binding constants or partition coefficients. As commonly known, a target is a target compound or a target receptor to which active ingredients or hazardous substances can bind. Particularly biological targets such as proteins, enzymes, antibodies, biological membranes or entire cells are of interest regarding the targets to be studied.

DE 198 14 775 discloses a method for determining binding constants and partition coefficients with regard to membranes. In that method, solid-supported membranes are brought into contact with an aqueous solution of the substance to be studied and incubated for a time sufficient for binding of the dissolved substance to the membrane surface. The solid-supported membranes are then separated from the solution (e.g., by sedimentation). The concentration of the substance to be studied is determined in the remaining solution (supernatant) or also, if applicable, in the separated solid-supported membranes by suitable measuring methods, and namely for a multiplicity of different quantity relations between solid-supported membrane and substance to be studied. For that purpose, either different amounts of the substance to be studied with respect to binding can be incubated in uniform solution volumes having in each case the same amount of solid-supported membrane or the substance to be studied in each case with identical concentration is added to the solid-supported membrane in different concentrations in uniform solution volumes. The total volume of the sample to be incubated, essentially composed of the volume of the buffer solution in which the substance to be studied or the substance mixture to be studied is dissolved and the volume of the carrier material including the membrane applied thereon, was always maintained at a constant level.

It could therefore be helpful to provide a method for determining interactions between a substance or a substance mixture and a target, with the method being improved over the method of DE 198 14 775.

### SUMMARY

I provide a method of determining interactions between a substance or a substance mixture and a target including incubating a first sample of the substance or of the substance mixture with the target immobilized on a solid, particulate carrier in a first buffer solution containing sample container, incubating a second sample of the substance or of the substance mixture with the target immobilized on a solid, particulate carrier in a second buffer solution containing sample container, separating the carrier from the buffer solution contained in the sample containers together with the target immobilized thereon and, if applicable, substance bound thereon, and measuring concentration of the substance or of the substance mixture in the respective supernatant, wherein the first and the second substance sample are incubated with different amounts of the target, and all sample containers include the same amount of buffer solution during incubation.

I also provide a device including first and second sample containers, each containing a target immobilized on a solid, particulate carrier substance as well as buffer solution, wherein the first and second sample containers include different amounts of the target, but the same amount of buffer solution.

### DETAILED DESCRIPTION

My method determines interactions between a substance or a substance mixture and a target. In this case, particularly biological membranes, proteins and nucleic acids come into consideration as targets. Similarly, synthetic or semi-synthetic receptors are of interest as targets, for instance lipid membrane model systems as described in DE 198 14 775. The substances to be studied or the substance mixtures to be studied can particularly be active ingredient molecules for pharmaceutical applications. Naturally, also the effects of hazardous substances and hazardous substance mixtures on biological targets such as cells can be studied.

The method thus comprises at least the following steps:
1. Incubating a first sample of the substance to be studied or the substance mixture to be studied with a target in a first buffer solution containing sample container. The target is always immobilized on a solid, preferably particulate carrier.
2. Incubating a second sample of the substance to be studied or the substance mixture to be studied with a target in a second buffer solution containing sample container. In this case as well, the target is always immobilized on a solid, preferably particulate carrier.
3. Separating the carrier together with the target immobilized thereon as well as, if applicable, substance bound thereon from the buffer solution contained in the sample containers. The buffer solution contains the portion of the substance to be studied or of the substance mixture to be studied that is not bound to the target.
4. Measuring the concentration of the substance or of the substance mixture in the respective supernatant, that is to say in the respectively remaining buffer solution.

In so doing, the first and the second sample are incubated with different amounts of the target, whereas however, during incubation all sample containers contain the same amount of buffer solution as well as preferably the same amount of the substance to be studied or the substance mixture to be studied, respectively. The result is that, in contrast to the above mentioned known procedure, the total volume of the samples to be incubated (volume of the buffer solution plus volume of the carrier including the target immobilized thereon) varies.

As mentioned above, the total volume composed of the buffer volume and the volume of the carrier material has always been maintained at a constant level in the method described in DE 198 14 775. An increased concentration of the carrier material resulted in a correspondingly decreased buffer volume. Surprisingly, I found that although the volume for the carrier material in relation to the buffer volume generally makes up for only a comparatively small part of the total volume, a corresponding decrease of the buffer volume may partially result in very significant errors regarding the determination of binding constants or partition coefficients. The reason for this is that the target-substance-binding process occurs as a function of concentration to a significantly higher extent than previously expected.

As a result of the procedure, by maintaining the amount of buffer solution at a constant level in the sample containers used, the source of errors could be overcome. In the outcome, my method provides substantially more reliable information on interactions between a substance or a substance mixture and a target than those obtained by conventional methods.

The carrier that the target is immobilized on is a carrier which cannot be dissolved in an aqueous solution. It can consist of organic or inorganic material whereby in the latter case, particularly metal oxides such as silicon dioxide and aluminum oxide, silicates, aluminates, borates or zeolites are to be mentioned. Metals and precious metals can generally be used as well.

As already mentioned, in a particularly preferred configuration, the carrier is particulate, that is to say in the form of particles. The particles usually have dimensions in the micrometer (μm) range, but at least partially they can also have dimensions in the range of nanometers (nm), as the case may be. The carrier, in particular the particles, can be nonporous or porous, with the latter case generally resulting in a significant increase of useful carrier surface (an “outer” surface formed by the exterior face of the carrier can be complemented, if applicable, by a useful “inner” surface in the form of the pore walls).

Targets can be immobilized on the outer and where appropriate also on the inner surface of the carrier by known means. Production of such carriers including immobilized targets is known and described, for example, in DE 100 48 822 related to lipid membrane model systems as targets.

Particularly preferably, lipid membranes or lipid bilayers are immobilized on a particulate carrier as targets. The targets surround the carrier particles preferably at least partially more preferred completely. Particularly preferably, the lipids are brain tissue lipids, that is to say lipids that can be found in brain tissues of animals or human beings. Preferably, the brain tissue lipids originate from brain tissue extracts. The corresponding lipids or extracts are known and commercially available from several companies.

Separation of the carrier can be effected, for instance, by filtration or centrifugation and, if required, subsequent decanting of the buffer solution. Particularly preferably, carrier particles having magnetic properties can be used. The particles can be separated from the buffer solution without any problems by applying a magnetic field.

Numerous analyzing methods are available to measure the concentration of the substance or of the substance mixture in the respective supernatant. In an exemplary manner, mass spectrometric or fluorescence spectroscopic methods may be mentioned, which may be coupled in a chromatographic method if required.

In particular, aqueous salt-based solutions are used as buffer solutions in a conventional way for biological applications and are commercially available.

Generally, a partition coefficient and/or a binding constant are determined from the measured substance concentrations to determine interactions between the substance studied or the substance mixture studied and the target. The correspondingly required mathematical instruments are known.

To determine a binding constant, at least two concentration values measured for different amounts of the target are required, which can be obtained by measuring the concentration of the substance or of the substance mixture in the supernatant of the first and the second sample containers. However, the error rate generally drops the more concentration values can be considered for determination purposes.

Thus, particularly preferably, in addition to the first and the second substance sample, at least one further sample of the substance or of the substance mixture, preferably between 1 and 10 further samples, with the target immobilized on a solid, preferably particulate carrier is/are incubated in at least one further buffer solution containing sample container, preferably in 1 through 10 further buffer solution containing sample containers. In this case, the further sample or further samples is/are incubated with different amounts of the target, respectively, and the amount in particular differ from the corresponding target amounts of the first and the second sample containers. However, during the incubation the sample containers always contain the same amount of buffer solution as the first and the second sample container.

It is preferred that the concentration of the substance or of the substance mixture in the respective supernatant is determined relatively to at least one reference sample. Such a reference sample is in particular a sample that exclusively contains buffer solution and the substance to be studied or the substance mixture to be studied.

Our devices comprise a first and a second sample container, in each case containing a target immobilized on a solid, particulate carrier substance as well as a buffer solution, wherein the first and the second sample containers include different amounts of the target, but the same amount of buffer solution.

The device can particularly be a microtiter plate. Correspondingly, the sample containers are preferably wells of a microtiter plate. In the wells, the substance samples can be incubated with a target. The method can particularly well be accomplished by a microtiter plate that includes at least a first and a second well which contain in each case a target immobilized on a solid particulate carrier substance (as described above) as well as buffer solution. According to the above explanations the first and the second well thereby contain different amounts of the target, but the same amount of buffer solution. Thus, the wells of the microtiter plate form the sample containers.

In most cases, common microtiter plates are rectangular and made from plastics such as polystyrene, polypropylene or polyvinyl chloride, or also glass in case of very special applications. They contain a multiplicity of wells isolated from one another which are usually arranged in rows and columns. The exact dimension (length×width×height) is preferably 127.76 mm×85.48 mm×14.35 mm. There is a multiplicity of formats, usually having the same surface area, but sometimes a variable height, so there are microtiter plates, for example, having
6 wells (2×3), filling volume in each case between 2 and 5 ml
12 wells (3×4), filling volume in each case between 2 and 4 ml
24 wells (4×6), filling volume in each case between 0.5 and 3 ml
96 wells (8×12), filling volume in each case between 0.3 and 2 ml
384 wells (16×24), filling volume in each case between 0.03 and 0.1 ml
1536 wells (32×48), filling volume in each case approximately 0.01 ml.
   The microtiter plate may be present in all configurations and form variations.

Preferably, besides the first and the second well, the microtiter plate includes at least one further well, preferably between 1 and 10 further wells filled with the same amount of buffer solution as the first and the second well.

The at least one further well or one or part of the further wells can be used as the previously mentioned reference. In this case, to practice the method the one or more wells is/are filled with the same amount of the substance to be studied or the substance mixture to be studied as the first and the second well. However, they do not contain any of the immobilized target.

Of course, the at least one further well or one or part of the further wells can also be used for determination of additional concentration values to reduce the mentioned error rate when determining a partition coefficient and/or a binding constant. In this case, besides the same amount of buffer solution as in the first and the second well, they contain even a predefined amount of the immobilized target which indeed must differ from the corresponding amounts in the first and the second well. To carry out the method, they are then also filled with the same amount of the substance to be studied or the substance mixture to be studied as the first and the second well.

A multitude of individual samples can generally be incubated in such a microtiter plate at the same time. Even determination of substance concentrations in the individual wells of such a plate can be effected simultaneously.

Thus, the individual steps of the method do not have to be carried out sequentially, particularly all incubation steps and/or concentration determinations are preferably effected simultaneously.

Also preferably, the sample containers are separate containers removably arranged on a support. The separate containers may, for example, be conventional Eppendorf tubes, the support may be designed as a support frame or a plate. Preferably, the support has the dimensions of a microtiter plate. Reference is made to the corresponding explanations as to possible dimensions of a microtiter plate. Furthermore, the separate containers may be vessels made of glass, particularly vials made from glass. For example, silanated glass containers are particularly suitable for test substances exhibiting strong affinity to plastic surfaces.

The above mentioned advantages and further advantages will become evident from the following description of a preferred example of the method. The individual features can thereby be realized as single features or as a combination thereof. The described examples merely serve for explanation purposes as well as for a better understanding and do not have a limiting character.

To determine the affinity of test substances to human serum albumin (HSA), 7 wells of a 96-well microtiter plate are filled as follows:

| | Well | | | | | | | | | | | | | |
|---|---|---|---|---|---|---|---|---|---|---|---|---|---|---|
| | 1 | | 2 | | 3 | | 4 | | 5 | | 6 | | 7 | |
| | Reference 1 | | V1 HSA | | V2 HSA | | V3 HSA | | V4 HSA | | V5 HSA | | Reference 2 | |
| HSA concentration | 0.0 | μL | 10.6 | μL | 20.2 | μL | 38.3 | μL | 72.9 | μL | 138.4 | μL | 0.0 | μL |
| Volume: HAS | 0.0 | μL | 8.8 | μL | 16.7 | μL | 31.8 | μL | 60.4 | μL | 114.8 | μL | 0.0 | μL |
| suspension on carrier | | | | | | | | | | | | | | |
| Volume: carrier | 0.0 | μL | 1.0 | μL | 1.9 | μL | 3.6 | μL | 6.8 | μL | 12.9 | μL | 0.0 | μL |
| Material | | | | | | | | | | | | | | |
| Volume: Buffer in | 0.0 | μL | 7.8 | μL | 14.9 | μL | 28.2 | μL | 53.6 | μL | 101.9 | μL | 0.0 | μL |
| suspension | | | | | | | | | | | | | | |
| Volume: Additional | 149.1 | μL | 141.3 | μL | 134.2 | μL | 120.9 | μL | 95.4 | μL | 47.2 | μL | 149.1 | μL |
| Buffer | | | | | | | | | | | | | | |
| Assay volume | 167.1 | μL | 167.1 | μL | 167.1 | μL | 167.1 | μL | 167.1 | μL | 167.1 | μL | 167.1 | μL |
| Buffer volume | 167.1 | μL | 167.1 | μL | 167.1 | μL | 167.1 | μL | 167.1 | μL | 167.1 | μL | 167.1 | μL |
| Total volume | 167.1 | μL | 168.1 | μL | 169.0 | μL | 170.7 | μL | 173.9 | μL | 180.0 | μL | 167.1 | μL |
| incl. carrier material | | | | | | | | | | | | | | |

The HSA was fixed on the surface of a carrier made of silica beads or silica pellets that were suspended in buffer solution (HSA suspension on carrier).

In each case, 18 μL of a 20 μM parent solution of a test substance were added to the wells. For example, desipramine was used as sample substance. After mixing by re-suspension, the silica beads/pellets were separated together with the HSA fixed thereon. The concentration of the test substance in the supernatants was determined by a mass spectrometric method (LCMS). The proportion of uncombined test substance in the wells including the HSA immobilized on the carrier was determined in relation to the references (calibration can be omitted by using the references, at least as long as the relation between signal and concentration for the test substances and the applied quantification system is linear).

The affinity of the test substance to human serum albumin (HSA) can be described by the following standard binding-model:

$${K}_{D} = \frac{[ A ] [ B ]}{[ AB ]}$$

wherein [A] refers to the concentration of uncombined test substance, [B] refers to the concentration of HSA and [AB] refers to the concentration of the HSA-test substance complex.

The concentration of uncombined test substance can also be described as the product of the relative proportion of uncombined test substance fᵤ and the sum of free and bound concentration of HSA:

[A]=fᵤ·([A]+[B]).

The following linear equation can be achieved by transformation:

$$\frac{{f}_{b}}{{f}_{u}} = \frac{1}{{K}_{D}} \cdot [ B ] .$$

In this equation, f_{b} refers to the relative proportion of the bound ligand.

By plotting the relation f_{b}/fᵤ against the receptor concentration [B], the binding parameter K_{D} can be determined exactly and in a robust manner. Outliers resulting from measurement techniques can particularly well be recognized by means of the method. To this end, the discordance method can be used for recognition of outliers in linear regression. Furthermore, the quality of the adaption of the measured data to the binding model can be well checked since the axis intercept of the regression model should be zero. Deviations thereof give notice of errors in the experiment, or they indicate that the simple non-cooperative binding model is not adequate to describe the data.

For further quality control of the adaption of the concentrations measured in the experiment to the binding model, the inverse value of the free concentration can be plotted against the ligand concentration. The axis intercept of this diagram corresponds to the reference concentration of the test system without a ligand. If the determined reference concentration corresponds to the reference concentration determined by measuring techniques, then a good model fitting is obtained.

The binding constant can then be determined from the regression of the f_{b}/fᵤ ratio against the free receptor concentration. The inverse value of the slope corresponds to the binding constant K_{D}.

The binding constants with respect to membranes can be determined according to the same principle if the molarity of the membrane lipids is known. As an alternative, the membrane affinity can be determined as a measure of the binding to membranes. The membrane affinity (MA) is defined as the relation between the concentration of a test substance in the lipid and the concentration of the test substance in the buffer:

$$MA = \frac{c ( lipid )}{c ( buffer )} .$$

From the total formula for the substance amount

nₜ=c(lipid)·V(lipid)·c(buffer)·(V(assay)−V(lipid))

a formula can be derived by means of which the membrane affinity can be determined experimentally:

$$\frac{{n}_{t}}{c ( buffer )} = ( \frac{c ( lipid )}{c ( buffer )} - 1 ) \cdot V ( lipid ) + V ( assay ) .$$

This is a linear relation as a function of the lipid volume. The membrane affinity corresponds to the slope of the regression model plus 1. Since the axis intercept of the regression model corresponds to the volume in the assay (buffer volume plus volume of the lipid), there is an internal quality control in this model as well.

To determine the membrane affinity MA of test substances to brain lipids, 7 wells of a 96-well microtiter plate were filled as follows:

| | Well | | | | | | | | | | | | | |
|---|---|---|---|---|---|---|---|---|---|---|---|---|---|---|
| | 1 | | 2 | | 3 | | 4 | | 5 | | 6 | | 7 | |
| | Reference 1 | | V1 Lipid | | V2 Lipid | | V3 Lipid | | V4 Lipid | | V5 Lipid | | Reference 2 | |
| Volume: lipid- | 0.0 | μL | 11.9 | μL | 21.2 | μL | 37.7 | μL | 67.1 | μL | 119.4 | μL | 0.0 | μL |
| suspension on a carrier | | | | | | | | | | | | | | |
| Volume: carrier | 0.0 | μL | 1.4 | μL | 2.6 | μL | 4.5 | μL | 8.1 | μL | 14.4 | μL | 0.0 | μL |
| Volume: lipid | 0.0 | μL | 0.1 | μL | 0.2 | μL | 0.4 | μL | 0.7 | μL | 1.2 | μL | 0.0 | μL |
| Volume: buffer in | 0.0 | μL | 10.3 | μL | 18.4 | μL | 32.8 | μL | 58.3 | μL | 103.8 | μL | 0.0 | μL |
| Suspension | | | | | | | | | | | | | | |
| Volume: additional | 146.4 | μL | 136.1 | μL | 128.0 | μL | 113.6 | μL | 88.1 | μL | 42.6 | μL | 146.4 | μL |
| Buffer | | | | | | | | | | | | | | |
| Assay- volume | 164.4 | μL | 164.5 | μL | 164.6 | μL | 164.8 | μL | 165.1 | μL | 165.6 | μL | 164.4 | μL |
| Buffer- volume | 164.4 | μL | 164.4 | μL | 164.4 | μL | 164.4 | μL | 164.4 | μL | 164.4 | μL | 164.4 | μL |
| Total volume | 164.4 | μL | 166.0 | μL | 167.2 | μL | 169.3 | μL | 173.2 | μL | 180.0 | μL | 164.4 | μL |
| incl. carrier material | | | | | | | | | | | | | | |

The brain lipids were fixed on the surface of the silica beads/pellets as carriers which were suspended in a buffer solution (lipid suspension on a carrier).

In each case 18 μL of a 20 μM parent solution of the test substance were added to the wells. After mixing by re-suspension the silica beads/pellets were separated together with the brain lipids fixed thereon. The concentration of the test substance in the supernatants was determined by a mass spectrometric method (LCMS). The quotient

nₜ/c(buffer)

of the test substance was then determined for all wells with lipid from the relative variation of the substance concentration in the wells 2 through 6 to the references 1 and 7. Calibration can be omitted as long as the relation between signal and concentration for the test substances and the applied quantification system is linear. The membrane affinity could be determined from the regression of the above quotient against the lipid volume.

1. A method of determining interactions between a substance or a substance mixture and a target comprising:
incubating a first sample of the substance or of the substance mixture with the target immobilized on a solid, particulate carrier in a first buffer solution containing sample container;
incubating a second sample of the substance or of the substance mixture with the target immobilized on a solid, particulate carrier in a second buffer solution containing sample container;
separating the carrier from the buffer solution contained in the sample containers together with the target immobilized thereon and, if applicable, substance bound thereon; and
measuring concentration of the substance or of the substance mixture in the respective supernatant,
wherein
the first and the second substance sample are incubated with different amounts of the target, and
all sample containers include the same amount of buffer solution during incubation.

2. The method according to claim 1, wherein a partition coefficient and/or a binding constant are determined from the substance concentration measured.

3. The method according to claim 1, wherein, in addition to the first and the second sample, at least one further sample of the substance or of the substance mixture are incubated with the target immobilized on a solid, particulate carrier in at least one further buffer solution containing sample container, wherein
the samples are incubated with different amounts of the target, and
all sample containers include the same amount of buffer solution during incubation as the first and the second sample container.

4. The method according to claim 1, wherein the concentration of the substance or of the substance mixture is determined in the respective supernatant relatively to a reference sample.

5. A device comprising first and second sample containers, each containing a target immobilized on a solid, particulate carrier substance as well as buffer solution, wherein the first and second sample containers include different amounts of the target, but the same amount of buffer solution.

6. The device according to claim 5, wherein the sample containers are wells of a microtiter plate.

7. The device according to claim 5, wherein the sample containers are separate containers removably arranged on a support, and said support has the dimensions of a microtiter plate.

8. The method according to claim 2, wherein, in addition to the first and the second sample, at least one further sample of the substance or of the substance mixture are incubated with the target immobilized on a solid, particulate carrier in at least one further buffer solution containing sample container, wherein
the samples are incubated with different amounts of the target, and
all sample containers include the same amount of buffer solution during incubation as the first and the second sample container.

9. The method according to claim 2, wherein the concentration of the substance or of the substance mixture is determined in the respective supernatant relatively to a reference sample.

10. The method according to claim 3, wherein the concentration of the substance or of the substance mixture is determined in the respective supernatant relatively to a reference sample.

11. The method according to claim 8, wherein the concentration of the substance or of the substance mixture is determined in the respective supernatant relatively to a reference sample.
